(19) United States
(12) Patent Application Publication
(10) Pub. No.: US 2011/0160166 A1
(43) Pub. Date: Jun. 30, 2011

(54) Therapeutic composition for the treatment and prevention of athlete's foot and fungal infections of the nail and surrounding tissues

(76) Inventor: Karen Hohenstein, West Hills, VA (US)

(21) Appl. No.: 12/651,410

(22) Filed: Dec. 31, 2009

(51) Int. Cl. A61K 31/60; A61P 31/02

(52) U.S. Cl. 514/159

(57) ABSTRACT

Methods and topical pharmaceutical formulations are provided for the treatment of athlete's foot (tinea pedis) and nail fungus (onychomycosis). The invention involves an anhydrous composition containing a therapeutically effective amount of salicylic acid. Repeated applications of the resulting formula over a 4-12 week period resolve most infections without the use of systemic drugs.

### TECHNICAL FIELD

This invention relates generally to methods and pharmaceutical formulations for treating athlete's foot and fungal infections of the nail and adjacent tissue.

### BACKGROUND

### Athlete's Foot (Tinea Pedis)

Athlete's foot (tinea pedis) is a fungal infection of the skin that causes itching, scaling, and cracking of affected areas. The majority of tinea pedis infections are caused by dermatophytes of the genus Trichophyton. The infection is communicable, and typically transmitted in public areas where people walk barefoot, such as swimming pools, gyms, or shower rooms. An estimated 70% of the adult population will have at least one athlete's foot infection annually. Many individuals suffer from recurrent or chronic infections. The open cracks in the skin of chronic sufferers are painful and may become infected by opportunistic bacteria or yeasts.

Current Treatments

Topical Treatments: Tinea Pedis is presently treated primarily with topical Over-the-Counter creams, powders or sprays containing antifungal agents such as clotrimazole, terbinafine, or tolnaftate. The user is typically directed to apply the medicament to the affected area once or twice daily for a period of one to four weeks. Tolnaftate-containing medicaments also direct once or twice daily use for the prevention of infections. Prescription antifungal creams, such as sertaconazole 2%, are rarely used to treat tinea pedis, primarily due to expense.

Current treatments for tinea pedis are only moderately effective and unpleasant or inconvenient. The sprays and powders in particular are messy to use and can cause stinging and burning sensations upon contact with open cracks in the affected skin. The majority of individuals who described themselves as chronic sufferers would prefer a daily topical treatment to cure infection and prevent new infection. The invention disclosed herein provides a novel treatment for tinea pedis that is effective, convenient and painless.

Nail Fungus (Onychomycosis)

Onychomycosis is a fungal infection of the nail and adjacent tissue. Onychomycosis is most commonly caused by several species of dermatophytes, primarily of the genera Trichophyton, Microsporum, and Epidermophyton. An estimated 6-8% of the adult population is affected. Infections can range from superficial, exhibiting unsightly discoloration, to severe, resulting in loss of the nail and deformity of the affected digit.

While not life threatening, onychomycosis infections cause significant impairment and embarrassment. Individuals with onychomycosis may have limited ability to perform routine tasks (e.g., picking up small objects, fastening buttons) and may be self-conscious and embarrassed by the unpleasant appearance of their nails.

Current Treatments

Topical Treatments: Ciclopirox 8% is the only FDA-approved topical treatment for nail fungus. It is not very effective: In clinical tests, fewer than 12% of patients achieved clear or almost clear nails after 48 weeks of daily treatment (Sanofi Aventis, Prescribing Information for Penlac® Ciclopirox 8%).

Oral Treatments: The preferred therapy for onychomycosis is orally administered terbinafine (Lamisil®, Novartis), itraconazole (Sporanox®, Ortho-McNeil), or fluconazole (Diflucan®, Pfizer). Terbinafine is an allylamine, effective against dermatophytes. Itraconazole and fluconazole are triazoles, effective against dermatophytes, nondermatophyte molds, and yeasts. When administered daily, these compounds carry the risk of hepatotoxicity, and monitoring of liver enzymes is advised. Terbinafine is an inhibitor of the Cytochrome P450 2D6 isozyme, and has several potentially serious interactions with commonly used 2D6-metabolized drugs such as tricyclic antidepressants, selective serotonin reuptake inhibitors, beta-blockers, antiarrhythmics class 1C and monoamine oxidase inhibitors Type B. The triazoles are inhibitors of Cytochrome P450 3A4 isozyme, and have several potentially serious interactions with commonly used 3A4-metabolized drugs such as statins, calcium channel blockers, oral hypoglycemics, benzodiazepines, and antiarrhythmics. In clinical studies of terbinafine, cure rates for toenail infections (12 weeks treatment, 36 weeks follow-up after completion of therapy) were 38%-59%. Clinical relapse rate was aproximately 15%. Terbinafine was more effective against fingernail infections. After 6 weeks of treatment, with assessment at 18 weeks after completion of therapy, the cure rates were 59-79% (Novartis, Prescribing Information for Lamisil® terbinafine 250 mg). In clinical studies of itraconazole, cure rates for toenail infections (12 weeks of daily treatment) were 14-35%, with a relapse rate of 21%. For fingernail infections, two pulses (one pulse=one week daily treatment, followed by three weeks of no treatment) produced cure rates of 47-56% with no relapse (Ortho-McNeil, Prescribing Information for Sporanox® itraconazole 200 mg).

Current treatments for onychomycosis result in limited success, carry significant risks of adverse effects and drug interactions, and are costly and inconvenient for the patient. A large majority of individuals suffering from onychomycosis would prefer a topical treatment rather than a systemic treatment with significant risk of adverse effects. It is also noted that prescription systemic treatments are expensive. A treatment program of terbinafine or itraconazole can cost between $400 and $1200, in addition to the costs of office visits to the prescribing physician. Most prescription drug insurance plans will not cover the cost of antifungal medications to treat onychomycosis. The invention disclosed herein anticipates a topical non-systemic treatment program costing under $25. The present invention provides a novel treatment for onychomycosis that is effective, safe, and convenient.

### SUMMARY OF THE INVENTION

It is a primary object of the invention to address the above needs in the art by providing a novel formulation for the treatment of athlete's foot (tinea pedis) and nail fungus (onychomycosis).

The invention provides a method for the treatment of tinea pedis and onychomycosis that involves a topically applied anhydrous formulation containing a therapeutically effective amount of salicylic acid. The formulation may be a paste or ointment, intended for long-term application to the body surface in the affected area.

### DETAILED DESCRIPTION OF THE INVENTION

### I. Definitions

Tinea pedis is a fungal infection of the feet. Three genera of fungal pathogens, Trychophyton, Epidermophyton, and Microsporum, are known to cause tinea pedis. The disease manifests as a pruritic, erythematous, scaly eruption on the foot. Painful interdigital cracking of the tissue may accompany the infection.

Onychomycosis is a fungal infection of the fingernail or toenail. The infection may encompass some or all of the surrounding tissues, including the nail bed, the nail matrix, the nail root, the nail folds, the cuticle, and the hyponychium. Infection may be caused by dermatophytes, nondermatophyte molds, yeasts, other fungi, or combinations of these.

The terms “treating” and “treatment” are used herein to refer to actions that reduce the severity and/or frequency of symptoms, eliminate symptoms and/or their underlying cause, prevent the occurrence of symptoms and/or their underlying cause, and improve or remediate damage. The present method of “treating” a subject, as the term is used herein, encompasses both prevention of tinea pedis and/or onychomycosis in a predisposed individual and treatment of tinea pedis and/or onychomycosis in a clinically symptomatic individual.

By “a therapeutically effective amount of salicylic acid” is meant a nontoxic but sufficient amount to provide the desired effect, i.e., treatment or prevention of athlete's foot and onychomycosis.

The term “topically applied” is used to mean application of a topical drug or pharmacologically active agent to the skin or nail.

The term “body surface” is used to refer to skin or nail.

### II. Pharmaceutical Formulations

The formulation may be in any form suitable for application to the nail and surrounding tissues, and may comprise an ointment or paste.

As known in the art of pharmaceutical formulation, ointments and pastes are semisolid preparations typically based on petrolatum or vegetable fats. The specific ointment base will provide for optimum drug delivery and be cosmetically acceptable to the user. An ointment base should be inert, stable, nonirritating and nonsensitizing. See Remington: The Science and Practice of Pharmacy, 21st Ed. (Lippincott Williams & Wilkins, 2005).

Salicylic acid USP is well known as a strongly antimicrobial pharmaceutical ingredient. It is currently approved by US FDA as an Over-the-Counter active ingredient in anti-dandruff and anti-acne medicaments. Salicylic acid is a skin and eye irritant and a keratolytic, and must be used at low concentrations when applied to human tissue. When formulated into an anhydrous base and applied to human nail and surrounding tissues, it was surprisingly found that the keratolytic and irritant effects were dramatically reduced or eliminated. Salicylic acid could be used at high concentrations for a strong, broad-spectrum antifungal effect to treat athlete's foot (tinea pedis) and nail fungus (onychomycosis) infections with no irritation experienced by the subject.

### III. Administration

The formulation of the invention may be applied to an area of body surface affected with tinea pedis and/or onychomycosis.

The treatment regimen will depend on a number of factors, such as the extent of the affected nail and/or tissue, and the responsiveness of the infection to treatment, but will normally be one or two doses per day, with a course of treatment lasting from several weeks to several months, until a cure or significant reduction of the infection is achieved.

With once-daily application of the instant invention, athlete's foot (tinea pedis) infections were resolved within one week. Open cracks in the skin healed within two to three weeks. Long term repeated use of the invention, for more than two years in some cases, has prevented new athlete's foot (tinea pedis) infections in previously chronic sufferers.

Nail fungus (onychomycosis) infections required longer treatment times. New healthy nail growth was observed after three to six weeks of once-daily application, although several months were required for full regrowth of uninfected healthy nail. When several subjects with long-established (one to two years or longer) nail fungus infections began treatment with the instant invention, they initially noticed a transitional zone of nail growth with visibly reduced infection. With continued treatment, completely clear, uninfected nail growth was observed within three months, and several additional months were required for complete regrowth of uninfected healthy nail.

The majority of subjects with nail fungus (onychomycosis) had infections of the toenails. Most subjects with fungus infections of the fingernail had developed the infection at a time when they used artificial acrylic fingernails. The application and maintenance of artifical nails require periodic visits to nail salons. All subjects with fingernail fungus infections believed they had acquired the infection during a visit to a nail salon. It should be noted that most subjects with fingernail fungus infections were under the treatment of a dermatologist and were also being treated with oral terbinafine, or had previously completed an 8-week oral terbinafine treatment cycle, but remained symptomatic. The instant invention was used as an adjunct treatment to the oral medication. In contrast, most subject with toenail fungus infections had not previously consulted a medical doctor for diagnosis or treatment.

### IV. Examples

The following non-limiting examples reflect several applications of the present invention. The practice of the present invention will employ conventional techniques of drug formulation, particularly topical drug formulation, which are within the skill of the art. Such techniques are fully explained in the literature. See Remington: The Science and Practice of Pharmacy, cited supra.

### Example 1

A topical ointment of the invention is prepared by conventional pharmaceutical methods. The indicated amounts of the following ingredients are used:

| INGREDIENT | AMOUNT |
|---|---|
| Petrolatum USP (Super White Protopet ®, Sonneborn) | 800 grams |
| Glyceryl Stearate (and) PEG-100 Stearate (Arlacel ® | 50 grams |
| 165, Croda) | |
| Salicylic Acid USP | 150 grams |

The Petrolatum USP and Glyceryl Stearate (and) PEG-100 Stearate are heated together until molten and uniform, approximately 75 degrees C. The mixture is stirred with cooling to a temperature of approximately 60 degrees C. The Salicylic Acid USP is then added, and the resulting mixture is stirred and cooled until it congeals into an ointment of the invention.

### Example 2

A topical ointment of the invention is prepared by conventional pharmaceutical methods. The addition of derivatized corn starch aids in the dispersal and suspension of the Salicylic Acid. The indicated amounts of the following ingredients are used:

| INGREDIENT | AMOUNT |
|---|---|
| Petrolatum USP (Snow White Petrolatum ®, Penreco) | 700 grams |
| Emulsifying Wax NF (Polawax ®, Croda) | 50 grams |
| Aluminum Starch Octenylsuccinate (Dry Flo ® Pure, | 100 grams |
| Akzo Nobel) | |
| Salicylic Acid USP | 150 grams |

The Petrolatum USP and Emulsifying Wax NF are heated together until molten and uniform, approximately 75 degrees C. The mixture is stirred with cooling to a temperature of approximately 60 degrees C. The Aluminum Starch Octenylsuccinate and Salicylic Acid USP are blended together until uniform, then added to the Petrolatum-Emulsifying Wax NF mixture. The resulting mixture is stirred and cooled until it congeals into an ointment of the invention.

### Example 3

A topical ointment of the invention is prepared by conventional pharmaceutical methods. A vegetable-sourced triglyceride blend replaces Petrolatum. The indicated amounts of the following ingredients are used:

| INGREDIENT | AMOUNT |
|---|---|
| Caprylic/Capric/Myristic/Stearic Triglyceride | 700 grams |
| (Softsan ® 378, Sasol) | |
| Emulsifying Wax NF (Polawax ®, Croda) | 50 grams |
| Corn Starch NF (Purity 21 ®, Akzo Nobel) | 100 grams |
| Salicylic Acid USP | 150 grams |

The Caprylic/Capric/Myristic/Stearic Triglyceride and Emulsifying Wax NF are heated together until molten and uniform, approximately 75 degrees C. The mixture is stirred with cooling to a temperature of approximately 65 degrees C. The Corn Starch NF and Salicylic Acid USP are blended together until uniform, then added to the Triglyceride-Emulsifying Wax NF mixture. The resulting mixture is stirred and cooled until it congeals into an ointment of the invention.

### Example 4

A topical ointment of the invention is prepared by conventional pharmaceutical methods. All ointment base ingredients are vegetable-sourced. The indicated amounts of the following ingredients are used:

| INGREDIENT | AMOUNT |
|---|---|
| Elaeis Guineensis (Palm) Oil (Lipovol ® | 700 grams |
| PAL, Lipo) | |
| PEG-40 Hydrogenated Castor Oil (Cremophor ® CO 40, | 50 grams |
| BASF) | |
| Tapioca Starch (Naviance ® P, Akzo Nobel) | 100 grams |
| Salicylic Acid USP | 150 grams |

The Palm Oil and PEG-40 Hydrogenated Castor Oil are heated together until molten and uniform, approximately 70 degrees C. The mixture is stirred with cooling to a temperature of approximately 60 degrees C. The Tapioca Starch and Salicylic Acid USP are blended together until uniform, then added to the oil mixture. The resulting mixture is stirred and cooled until it congeals into an ointment of the invention.

The purpose of the above description and examples is to illustrate certain embodiments of the present invention without implying any limitation. It will be apparent to those of skill in the art that various modifications may be made to the composition and method of the present invention without departing from the spirit or scope of the invention.

All publications mentioned herein are hereby incorporated by reference in their entireties.

I claim:

1. A composition consisting of a pharmaceutically and cosmetically acceptable anhydrous base, containing a therapeutically effective amount of salicylic acid.

2. A method of treatment of tinea pedis, comprising topically applying to the affected tissues the formulation of claim 1.

3. A method of treatment of onychomycosis, comprising topically applying to the affected nail and surrounding tissues the formulation of claim 1

4. A method of prevention of tinea pedis, comprising topically applying the formulation of claim 1 to the skin at the base of the toes.

5. A method of prevention of onychomycosis, comprising topically applying the formulation of claim 1 to the nails.

6. The composition of claim 1, in which the base consists of petrolatum.

7. The composition of claim 1, in which the base consists of petrolatum and high HLB surfactants to form a water-dispersible ointment.

8. The composition of claim 1, in which the base consists of vegan petrolatum substitute.

9. The composition of claim 1, in which the base consists of vegan petrolatum substitute and high HLB surfactants to form a water-dispersible ointment.

10. The composition of claim 1, in which the salicylic acid concentration is in the range of 5-50% w/w.

11. The method of claim 2, wherein the formulation is applied periodically over an extended time period.

12. The method of claim 2, wherein the formulation is applied once daily.

13. The method of claim 2, wherein the formulation is applied twice daily.

14. The method of claim 2, wherein said extended time period is at least three months.

15. The method of claim 2, wherein said extended time period is at least six months.

16. The method of claim 3, wherein the formulation is applied periodically over an extended time period.

17. The method of claim 3, wherein the formulation is applied once daily.

18. The method of claim 3, wherein the formulation is applied twice daily.

19. The method of claim 3, wherein the formulation is applied on an as-needed basis.

20. The method of claim 4, wherein the formulation is applied periodically over an extended time period.

21. The method of claim 5, wherein the formulation is applied periodically over an extended time period.
